(19) United States
(12) Patent Application Publication
(10) Pub. No.: US 2025/0195921 A1
(43) Pub. Date: Jun. 19, 2025

(54) METHOD AND SYSTEM FOR SUPPORTING THE PROCESS OF POSITIONING A PATIENT

(71) Applicant: Lap GmbH Laser Applikationen, Lüneburg (DE)

(72) Inventors: Daniel KAYSER, Hamburg (DE); Thomas Speck, Mannheim (DE)

(21) Appl. No.: 18/858,271

(22) PCT Filed: Apr. 3, 2023
(86) PCT No.: PCT/EP2023/058683

§ 371 Date: Oct. 18, 2024

(30) Foreign Application Priority Data
Apr. 19, 2022 (DE) 10 2022 109 402.2

(51) Int. Cl. A61N 5/10

(52) U.S. Cl. CPC A61N 5/1049; A61N 5/1037; A61N 2005/105

(57) ABSTRACT

A system for supporting a process of positioning a patient in a treatment room includes a 3D surface detection system to detect a surface of a patient's body and an evaluation apparatus that determines (i) at least one point of intersection between at least two lines and the surface of the patient's body and (ii) a reference surface of the patient's body indicating a target position of the patient's body relative to an isocenter of the irradiation device. The evaluation apparatus determines a transformation value required to match the detected surface with the reference surface. A target position of the at least one point of intersection is determined by the evaluation apparatus based on the transformation value. A visualization system displays the at least one point of intersection and the target position thereof. The at least one point of intersection indicates the isocenter of the irradiation device.

### CROSS-REFERENCE TO RELATED APPLICATIONS

This application is a national stage application pursuant to 35 U.S.C. § 371 of International Application No. PCT/EP2023/058683, filed on Apr. 3, 2023, which claims priority to, and benefit of, German Patent Application No. 10 2022 109 402.2, filed Apr. 19, 2022, the entire contents of which are hereby incorporated by reference.

### TECHNOLOGICAL FIELD

The following disclosure is directed to a method and a system for supporting the process of positioning a patient in a treatment room for an irradiation process with an irradiation device. During radiation therapy, tumors, for example, are irradiated with such irradiation devices. Linear accelerators (linacs), among other devices, are used as irradiation devices. An important aspect of radiation therapy is the precise positioning of the patient so that the dose of radiation generated by the irradiation device hits the body tissue to be treated as precisely as possible.

### BACKGROUND

Positioning the patient using lasers and markings on the patient's skin is known. Such a system is known, for example, from DE 195 24 951 A1. Laser systems for projecting laser planes for patient positioning, in particular coronal, transverse, and sagittal planes, exist, for example, in examination rooms with imaging devices, for example, CT scanners for CT simulation, and on irradiation devices. For example, in the course of a CT simulation and subsequent radiation planning, the isocenter of the tumor to be treated in the body of the patient is determined using imaging. At a later point in time, this isocenter of the tumor will be matched as precisely as possible to the isocenter of the irradiation device, typically in a separate treatment room. In order to make the position of the isocenter of the tumor visible on the patient's body from the outside, at least three laser planes that are orthogonal to each other are aligned with the imaging system in the examination room so that their common point of intersection is at the isocenter of the tumor. For this alignment, the laser planes can be shifted in the room with movable laser rails and/or the patient can be moved in the room on a patient bed. The projected laser planes are shown as lines on the surface of the patient's body, wherein each two laser lines form a point of intersection on the surface of the body. In this manner, for example, three points of intersection, each formed by two of the three orthogonal laser planes, can be indicated on the surface of the patient's body, for example, on two opposite sides and on the upper side of the patient's body. The points of intersection of the laser lines can be marked by means of pens or markings applied to the skin. During a subsequent irradiation in the treatment room, the isocenter of the irradiation device can in turn be indicated on the surface of the patient's body, using three orthogonally aligned laser planes, by the point of intersection of the three lines in the room. To align this isocenter of the irradiation device with the isocenter of the tumor, the patient can be shifted or, respectively, rotated, for example, by means of a movable patient bed, in the room so that the points of intersection on the surface of the patient's body, each formed by two of the orthogonal laser planes match with the previously applied markings. When all the points of intersection on the surface of the body have been brought into alignment with the previously applied markings, the positioning of the patient is completed and the irradiation can begin.

This method of positioning the patient is easy to perform and intuitive for the user, since it can be seen immediately which movement of the patient is required to bring the laser lines and the markings on the surface of the patient into alignment and thus bring the patient into the desired position. A disadvantage of this method is that the patient must bear the markings on their body at least for the duration of the irradiation treatment, usually for multiple weeks. There is the risk of markings being washed off or otherwise becoming unrecognizable, whereby positioning the patient is no longer possible with sufficient precision. Changes in the patient's body during the irradiation treatment, for example, weight loss or a change in the rigidity or shape of the patient's body, also cannot always be recognized and taken into account sufficiently precisely. In addition, only three selected points are used for positioning the patient, which should also be as precise as possible even outside of the plane of the points of intersection.

3D surface detection systems are also known from the prior art, which allow a positioning of the patient for radiation therapy without markings on the body. Surface detection systems are described, for example, in U.S. Pat. Nos. 7,348,974 B2, 7,889,906 B2, 9,028,422 B2, US 2015/265852 A1, and US 2016/129283 A1. In surface detection systems, a three-dimensional surface of the patient's body is measured live and compared with a three-dimensional reference surface of the patient's body created, for example, previously as part of radiation planning, for example, using a CT simulation. Deviations are displayed in three-dimensional visualizations of the target and the actual surfaces. Values for required shifts and rotations of the patient that must be applied to the position of the patient can also be calculated so that the target and actual surfaces match. The accuracy of the positioning of the patient is often greater with such three-dimensional surface detection systems because the alignment takes place not only along projected lines or intersection points, but based on entire surfaces or portions of the surface. However, the interpretation of the values displayed by the system in relation to the positioning of the patient is less intuitive and requires a greater period of familiarization and learning on the part of the user. This can lead to user errors.

Starting from the explained prior art, the object of the invention is therefore to provide a method and a system of the type mentioned at the outset with which the positioning of a patient for irradiation at an irradiation device is supported in a manner that is simple and intuitive for the user and at the same time provides the greatest possible precision.

### BRIEF SUMMARY OF THE INVENTION

Aspects of the following disclosure are directed to embodiments of a method for positioning a patient in a treatment room for an irradiation process/treatment. In some embodiments, the method includes detecting a current surface of the patient's body, preferably in the treatment room, using a 3D surface detection system. At least one point of intersection between at least two lines and the detected surface of the patient's body is calculated. The at least one point of intersection indicating the isocenter of the irradiation device using a reference surface of the patient's body. The reference surface indicates the target position of the patient's body relative to the isocenter of the irradiation device, in order to calculate a transformation required to produce a match between the detected surface of the patient's body and the reference surface. In some embodiments, a target position of the at least one point of intersection with the detected surface of the patient's body is determined. The at least one point of intersection indicates the isocenter of the irradiation device, on the basis of the calculated transformation, displaying the at least one point of intersection with the detected surface of the patient's body, said point of intersection indicating the isocenter of the irradiation device, and the target position thereof in a visualization system.

Aspects of the following disclosure are directed to embodiments of a system for positioning a patient in a treatment room for an irradiation process/treatment including a 3D surface detection system configured to detect a current surface of the patient's body, preferably in the treatment room. Some embodiments of the system further include an evaluation apparatus configured to calculate at least one point of intersection between at least two lines and the detected surface of the patient's body, said point of intersection indicating the isocenter of the irradiation device. In some embodiments, the evaluation apparatus is further configured, using a reference surface of the patient's body, said reference surface indicating the target position of the patient's body relative to the isocenter of the irradiation device, to calculate a transformation required to produce a match between the detected surface of the patient's body and the reference surface. In some embodiments, the evaluation apparatus is further configured to determine a target position of the at least one point of intersection with the detected surface of the patient's body, said point of intersection indicating the isocenter of the irradiation device, on the basis of the calculated transformation. In some embodiments, the system further includes a visualization system configured to display the at least one point of intersection with the detected surface of the patient's body, said point of intersection indicating the isocenter of the irradiation device, and the target position thereof on the basis of the calculated transformation.

In some embodiments, the irradiation device serves to irradiate a patient, for example, to treat a tumor. The device can be a linear accelerator (linac). According to the invention, the position of the patient, who is preferably located in the treatment room, for example, on a movable patient bed, is detected with a three-dimensional surface detection system. The three-dimensional surface of the patient's body is detected in particular multiple times in succession, for example, at fixed time intervals or continuously. The three-dimensional surface detection system can be designed in a manner known per se. It can comprise, for example, cameras, in particular a stereoscopic camera system, as known per se.

In some embodiments, the dose of radiation generated by the irradiation device is often the greatest in the isocenter of the irradiation device. The isocenter of the irradiation device should match as precisely as possible with the isocenter of the tissue to be irradiated, in particular a tumor. For this purpose, according to the invention a point of intersection between at least two lines or, respectively, planes on the surface of the body of the patient is calculated. This point of intersection indicates the isocenter of the irradiation device. It is calculated from the lines of intersection of orthogonal planes with the surface of the body, said lines intersecting in the isocenter of the irradiation device. The isocenter as a point of intersection between, for example, three orthogonal planes typically lies inside the patient's body positioned on the patient bed. The, for example three, orthogonal planes are therefore shown as three lines on the patient's body, each pair of lines intersecting on the surface of the body, for example on two opposite sides and the upper side of the body. The points of intersection between each two of the three lines and the surface of the body thus indicate the isocenter. These lines and their points of intersection can be calculated and visualized according to the invention. Accordingly, three points of intersection between each two lines and the detected surface of the patient's body can be calculated, namely the points of intersection of the three orthogonal planes intersecting in the isocenter of the irradiation device at three positions on the surface of the patient's body. Of course, more than three points of intersection on the surface of the body can also be calculated and visualized, for example, four points of intersection, if, for example, a point of intersection on the underside of the patient's body is also calculated. If lines and their points of intersection are discussed in the present case, this also accordingly comprises the (orthogonal) planes that are shown as lines on the surface of the body.

In some embodiments, the invention also takes into account a three-dimensional reference surface of the patient's body, which indicates the target position of the patient's body relative to the isocenter of the irradiation device. The reference surface specifies the target position of the patient's body for the irradiation. The reference surface can be created, for example, as part of irradiation planning preceding the irradiation. To do so, the tissue to be irradiated, in particular the tumor, can be determined with an imaging method, such as a CT or MRI method. The surface of the patient's body can be detected, for example, in an examination room with a 3D surface detection system and it can be calculated accordingly how this surface must be aligned in relation to the isocenter of the irradiation device for the subsequent irradiation. The 3D surface detection system in the examination room can in principle be designed in the same way as the 3D surface detection system in the treatment room.

On the basis of the reference surface, which is created in this way or in another way, for example, from data from an imaging method, a transformation of the current surface of the patient's body detected by the 3D surface detection system, in particular in the treatment room, is calculated in order to produce a match between the current surface and the reference surface, in particular such that the isocenter of the irradiation device matches with the center of the tissue to be irradiated in the patient's body. The transformation can comprise, for example, a required shift in all three spatial directions and/or a rotation of the patient's body and/or of the reference surface about one or more axes of rotation. The transformation can be calculated, for example, with iterative methods that are known per se, such as the iterative closest point method.

According to some embodiments, a target position of the at least one point of intersection with the surface of the patient's body, said point of intersection indicating the isocenter of the irradiation device, is also determined on the basis of the calculated transformation. The at least one point of intersection with the detected surface of the patient's body, said point of intersection indicating the isocenter of the irradiation device, and the target position thereof determined on the basis of the transformation are displayed in a visualization system. On the basis of the transformation or, respectively, an inverse transformation performed on this basis, a target position in the coordinate system of the patient is determined for the previously calculated point of intersection indicating how it would be shown on the patient's body if the patient's body were aligned as specified relative to the isocenter of the irradiation device. The target position thereby corresponds to at least one point on the reference surface of the patient's body in the coordinate system of the patient. On the basis of the transformation or, respectively, inverse transformation calculated according to the invention, such a target position can be determined in principle for any points on the surface of the patient's body, meaning, inter alia, the point of intersection of the lines or, respectively, orthogonal planes indicating the isocenter. On the basis of the display of the point of intersection and the target position in the visualization system, a user can easily position the patient such that the point of intersection matches the target position thereof and the patient is aligned as specified relative to the isocenter of the irradiation device. The visualization system is software-based. It can be executed, for example, on a PC, laptop, notebook, tablet, or the like. The visualization takes place accordingly on a display of a corresponding device.

In some embodiments, if the surface or the reference surface of the patient's body is discussed in the present case, this comprises in principle the surface or, respectively, reference surface of the patient's entire body or only a portion of the patient's body, for example, a portion of the patient's body that is of interest for the irradiation. It should be further noted that the method according to the invention is preferably performed in the treatment room with the irradiation device, or, respectively, that the system according to the invention is preferably located in the treatment room. However, this is not strictly required. Additionally or alternatively, the method could also be performed in a different room or, respectively, the system could be located in a different room, for example, in an examination room. For example, the method according to the invention could already be performed in an examination room before the positioning of the patient in a treatment room.

In some embodiments, the invention is based on the idea of calculating virtual markings on the detected surface of the patient's body and virtual positioning lasers using a currently detected three-dimensional surface of the patient's body and a transformation of the patient's body calculated using a three-dimensional reference surface and representing them to the user. The positioning of the patient can be intuitively and easily performed on the basis of the virtual markings and virtual positioning lasers. At the same time, the advantages of the highly precise surface detection and the transformation determined therefrom are utilized. This achieves the precision of 3D surface detection systems with simple and intuitive operation, as in the case of classic markings on the surface of the body and corresponding positioning lasers. The advantages of both systems are combined without the corresponding disadvantages. The preferably continuous calculation and display of the at least one point of intersection and the target position thereof on the surface of the body visualizes for the user in a simple manner the degrees of freedom and directions in which the patient needs to be moved in order to produce a match between the at least one point of intersection and the target position thereof.

In some embodiments of the method according to the invention, the steps thereof can be performed multiple times in succession, for example, at fixed time intervals or preferably continuously. In this manner, an ongoing live detection of the position of the patient and required repositioning of the patient for the irradiation can be displayed in the visualization system and can be checked by the user. The user can thus successively approach and/or monitor the correct patient position in a simple manner. In some embodiments, to simplify the positioning, the at least one point of intersection and the target position thereof can be displayed in the visualization system as crosses of lines.

According to some embodiments, a patient bed bearing the patient's body and/or the patient on the patient bed can be moved in the treatment room so that the at least one point of intersection with the detected surface of the patient's body, said point of intersection indicating the at least one isocenter of the irradiation device, matches with the target position thereof. The positioning can be controlled automatically or manually. For this purpose, the patient's body can be located on a movable patient bed, which can be moved, for example, along three directions that are perpendicular to each other and can also be rotated about one or more axes of rotation, for example, a vertical axis of rotation and an axis of rotation along a longitudinal and/or transverse direction of the patient bed. Thus, the user can position the patient bed or, respectively, the patient so that the markings of the isocenter of the irradiation device match in the visualization system with the target position of the markings.

According to some embodiments, it can be provided that at least three points of intersection between at least three lines and the detected surface of the patient's body, said points of intersection indicating the isocenter of the irradiation device, are calculated and displayed in the visualization system, and that at least three target positions, determined on the basis of the calculated transformation, of the at least three points of intersection with the detected surface of the patient's body, said points of intersection indicating the isocenter of the irradiation device, are displayed in the visualization system. As already explained, the isocenter of the irradiation device can be defined by the point of intersection of three orthogonal planes in particular in the treatment room. Since this isocenter is typically located within the patient's body, each two of the orthogonal planes, as lines depicted on the surface of the body, form a point of intersection on the surface of the patient's body, wherein a total of three points of intersection are formed by three orthogonal planes, for example, on opposite sides of the patient's body and on the upper side of the patient's body. According to the aforementioned embodiment, these points of intersection can be calculated and displayed in the visualization system. Accordingly, on the basis of the transformation calculated according to the invention, the target positions of these points of intersection can also be calculated in the manner explained above and displayed in the visualization system. The positioning of the patient's body is further improved by this embodiment in that all points of intersection must be brought into alignment with their target positions by a corresponding travel movement of the patient bed and/or movement of the patient's body on the patient bed.

According to some embodiments, the detected surface of the patient's body can also be represented in the visualization system, wherein the at least one point of intersection with the detected surface of the patient's body, said point of intersection indicating the isocenter, and the target position thereof are displayed on the surface of the patient's body represented in the visualization system. Of course, the same applies to any other points of intersection. The representation of the patient's body together with the points of intersection and the corresponding target positions on its surface further simplifies the visualization and thus the positioning of the patient for the user.

According to some embodiments, the at least two lines defining the point of intersection indicating the isocenter of the irradiation device can also be displayed in the visualization system. The target position of the at least one point of intersection indicating the isocenter of the irradiation device can also be displayed in the visualization system by the point of intersection of at least two lines. In particular, the lines can be displayed on the currently detected surface of the patient, which is also represented in the visualization system. By displaying and thus aligning the lines during the positioning of the patient, the positioning accuracy is further improved in that longer lines, which can extend in particular over the entire length and/or width of the patient's body, must be made to match. Thus, substantially a complete virtual simulation of the positioning of the patient described above in the prior art takes place using projected orthogonal laser planes and their intersections, represented by lines on the surface of the patient, with same.

According to some embodiments, the reference surface of the patient's body can, be created as part of radiation planning preceding the positioning of the patient. The reference surface can be created in particular in an examination room separate from the treatment room. In a manner known per se, it can comprise an imaging system, for example, a CT or MRI system. The patient's body is detected together with the tissue to be irradiated, for example, a tumor. On this basis, the reference surface can be created in a manner known per se.

According to some embodiments, additional points of intersection with the detected surface of the patient's body beyond the points of intersection indicating the isocenter of the irradiation device can be calculated. The additional points of intersection can then be displayed in the visualization system together with their target positions determined in turn on the basis of the calculated transformation. The virtual marking and alignment using additional points of intersection, for example, distinctive points on the surface of the body such as hip bones, skull, nose, feet, knees, etc., can further improve the positioning of the patient in the room. Similarly to a mannequin, for example, points can be marked on the surface that make a movement of the patient's body or a deformation of the patient's body visible, for example, in the abdominal region or the like. In particular a change in the shape of the body, for example, when the rigidity of the patient's body changes, thus becomes visible. This would not always be reliably recognized, for example, if only the isocenter was marked. The additional points of intersection can also each be points of intersection of two lines or, respectively, orthogonal planes. In turn, the lines defining the additional points of intersection can also be represented on the surface of the body in the visualization system. Since according to the invention the surface of the patient's body is detected and a transformation is calculated for this surface, in principle any points can be marked and indicated on the surface of the body. In practice, a suitable compromise is selected between the number of points to be indicated and thus brought into alignment by moving the patient bed and/or moving the patient's body on the patient bed, on the one hand, and the required positioning accuracy, on the other hand.

In some embodiments, to further improve the positioning of the patient, the additional points of intersection can be, for example, points of intersection with edge regions of the surface of the patient, for example on opposite ends of the patient's body. A particularly precise positioning is possible in that markings that are far away from each other must be brought into alignment with their respective target positions.

According to some embodiments, the at least one point of intersection and the target position thereof can be displayed from various angles in the visualization system. This further simplifies the positioning. A display can take place, for example, from opposite sides of the patient's body and from an upper side of the patient's body. This applies accordingly to the three-dimensional surface of the patient's body if it is also displayed in the visualization system.

According to some embodiments, the target position of the at least one point of intersection with the detected surface of the patient's body, said point of intersection indicating the isocenter of the irradiation device, can be verified using line or cross lasers arranged in the treatment room and/or an examination room. In this embodiment, physical lasers that project the orthogonal laser planes in the room and are arranged in the treatment room or, respectively, the examination room are used to define the position of the virtual laser markings. For this purpose, the current position of the physical lasers located in the treatment room or, respectively, examination room can be detected and transferred to the virtual surface data of the system according to the invention.

According to some embodiments, the at least one additional target position of at least one additional point of intersection with the surface of the patient's body can be determined, and the at least one additional point of intersection with the detected surface of the patient's body and the additional target position thereof can be displayed in the visualization system. In this manner, additional virtual points of intersection with the detected surface of the patient's body can be determined and adopted as the target position.

According to some embodiments, a position of a point of intersection can be indicated on the detected surface of the patient's body with line or cross lasers arranged in the treatment room and/or an examination room, and the indicated position can be determined as the target position or as an additional target position and displayed in the visualization system. Additional points of intersection with the detected surface of the patient's body can thus be indicated by physical line or cross lasers and read out by the system according to the invention. These additional points of intersection can then be adopted as (additional) target positions.

### BRIEF DESCRIPTION OF THE DRAWINGS

An exemplary embodiment of the invention is explained below in greater detail with reference to figures.

FIG. 1 schematically illustrates an embodiment of a system according to the invention.

FIG. 2 schematically illustrates a representation in a visualization system of the system according to the invention in a first operating state.

FIG. 3 schematically illustrates the representation from FIG. 2 in a second operating state.

FIG. 4 schematically illustrates the representation from FIG. 2 in a third operating state.

The same reference signs refer to the same objects in the figures unless indicated otherwise.

### DETAILED DESCRIPTION OF THE INVENTION

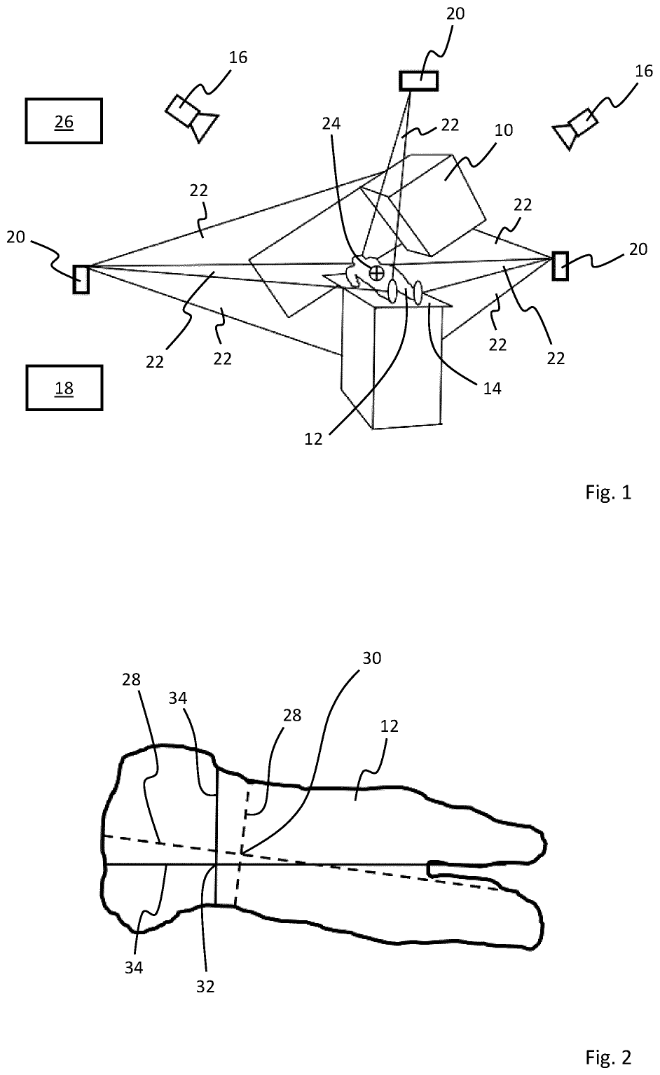

FIG. 1 shows the system according to the invention in a treatment room with an irradiation device 10, in which the irradiation device can be, for example, a linear accelerator (linac) for tumor treatment. A patient's body 12 is located on a movable patient bed 14 in the treatment room, which can be moved, for example, along the three spatial directions and can be rotated about a vertical axis. It is also possible for the patient bed 14 to be rotatable about its longitudinal and/or transverse axis. The irradiation device 10 can be rotated about the patient's body 12 during irradiation in a manner known per se.

The system also comprises a 3D surface detection system with two stereoscopic cameras 16 in the example shown. The current three-dimensional surface of the patient's body 12 is detected in the treatment room with the cameras 16. This can take place multiple times in succession, in particular continuously. The detected surface of the patient's body 12 is represented in a visualization system 18 of the system according to the invention, as explained in more detail in the following.

Furthermore, in the treatment room, three line or cross lasers 20 are arranged, which project at least three laser planes 22 that are orthogonal to each other in the room and intersect each other in the isocenter 24 of the irradiation device 10. This isocenter 24 is regularly located inside the patient's body 12. The laser planes 22 projected by the line or cross lasers 20 are accordingly depicted in the shape of lines on the surface of the patient's body 12, wherein each two of the laser lines depicted on the surface of the body form a point of intersection on the surface of the patient's body 12. For example, three points of intersection can thus be formed on the surface of the patient's body, namely on opposite sides, in FIG. 1 the left and right side, of the patient's body 12 and on the upper side of the patient's body 12.

The system according to the invention also comprises an evaluation apparatus 26, which, independently of the line or cross lasers 20 physically present in the treatment room, calculates the points of intersection, indicating the isocenter 24 of the irradiation device 10, between each of at least two lines, corresponding to the planes projected by the physical line or cross lasers 20, and the surface of the patient's body 12 detected by the 3D surface detection system, in particular the cameras 16. For example, as part of radiation planning, preceding the irradiation, a three-dimensional reference surface of the patient's body is also created with an imaging system, such as a CT or MRI system, which indicates the target position of the patient's body 12 in relation to the isocenter 24 of the irradiation device 10. Using this reference surface, a transformation required to match the surface of the patient's body 12 detected by the 3D surface detection system with the reference surface is calculated by the evaluation apparatus 26. On the basis of the calculated transformation, a target position of the points of intersection with the surface of the patient's body 12, said points of intersection indicating the isocenter 24 of the irradiation device 10, is in turn determined by the evaluation apparatus 26. Typically, the target position of the patient's body 12 is such that the isocenter of the tissue to be treated, in particular the tumor to be irradiated, coincides with the isocenter 24 of the irradiation device 10.

The points of intersection with the detected surface of the patient's body 12, said points of intersection indicating the isocenter 24 of the irradiation device 10, and the respective target positions thereof are displayed in the visualization system 18. This will be explained in more detail based on FIGS. 2 to 4.

FIG. 2 shows a case in which the patient's body 12 is not yet correctly aligned. In the example shown, in addition to the detected patient's body 12, the lines 28 that define the points of intersection indicating the isocenter of the irradiation device are shown in the visualization system 18. In FIG. 2, the point of intersection 30 is defined by the lines 28 as a point of intersection formed on the upper side of the patient's body 12. Additional points of intersection are typically formed on opposite sides of the patient's body. As can be seen in FIG. 2, the target position 32 of the point of intersection 30 is also displayed in the visualization system 18, namely also as point of intersection 32 of two lines 34, meaning target lines 34. It can be seen in FIG. 2 that the patient's body is still not correctly positioned for the irradiation. The point of intersection 30 and the target position 32 thereof as well as the lines 28, 34 defining it are clearly not yet in alignment.

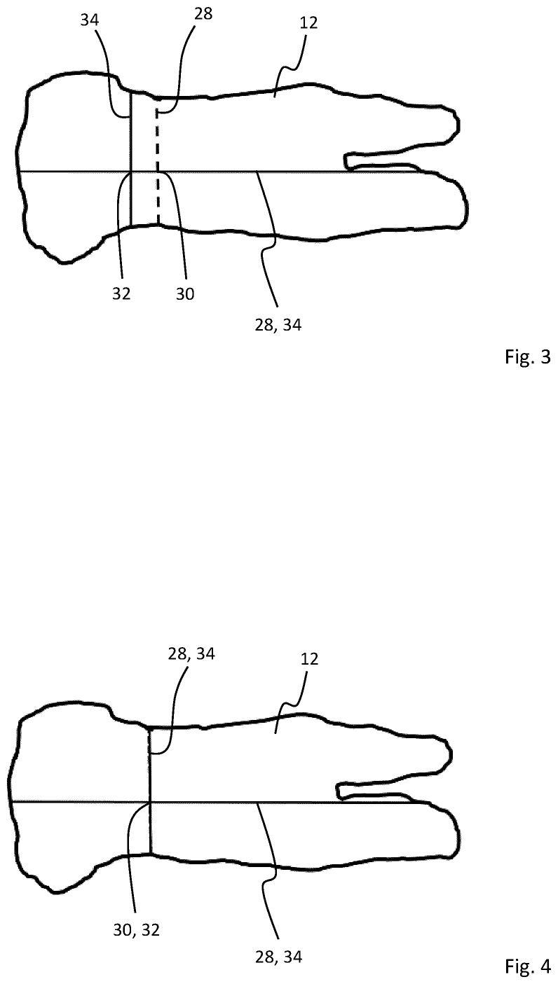

In FIG. 3, a first step of the positioning of the patient has taken place. In particular, a rotation of the patient's body 12 and/or of the patient bed 14 about a vertical axis of rotation has taken place so that the shift that can still be seen in FIG. 2 between the lines 28 and 34 is remedied. The lines 28, 34 running in the longitudinal direction are already aligned with each other. There is still a distance between the lines 28, 34 running transversely to the longitudinal direction of the patient's body 12, and this distance also exists accordingly between the point of intersection 30 and the target position 32 thereof.

In FIG. 4, this distance is also remedied in that the patient's body 12 has been shifted by correspondingly moving the patient bed 14 and/or moving the patient's body on the patient bed 14 in the longitudinal direction so that now all lines 28, 34 and thus the point of intersection 30 and the target position 32 thereof are in alignment.

The alignment of the additional points of intersection of the lines defining the isocenter 24 of the irradiation device 10 takes place in a corresponding manner for the complete positioning of the patient's body 12. The positioning of the patient is completed and the irradiation can take place.

It is also possible to calculate and represent additional points of intersection of corresponding lines on the surface of the patient's body 12. In this manner, the positioning accuracy can be further improved. In the visualization system 18, the patient's body 12 with the points of intersection 30, 32 and the lines 28, 34 can be displayed accordingly from different angles to simplify aligning all points of intersection.

If desired, the alignment of the calculated lines 28, 34 represented in the visualization system 18 can be verified by means of the physical line or cross lasers 20 arranged in the treatment room.

### LIST OF REFERENCE SIGNS

10 Irradiation device
12 Patient's body
14 Patient bed
16 Cameras
18 Visualization system
20 Line or cross lasers
22 Laser planes
24 Isocenter of the irradiation device
26 Evaluation apparatus
28, 34 Lines
30 Point of intersection
32 Target position of the point of intersection

1-24. (canceled)

25. A method for supporting a process of positioning a patient's body in a treatment room for an irradiation process with an irradiation device, comprising:
detecting a surface of the patient's body using a 3D surface detection system;
calculating at least one point of intersection between at least two lines and the surface of the patient's body, wherein the point of intersection corresponds to an isocenter of the irradiation device;
determining a reference surface indicating a target position of the surface of the patient's body relative to the isocenter of the irradiation device;
determining a transformation value for the current surface of the patient's body to match the reference surface;
determining a target position of the at least one point of intersection with the surface of the patient's body based on the transformation value, wherein the at least one point of intersection indicates the isocenter of the irradiation device; and
displaying the at least one point of intersection with the surface of the patient's body and the target position thereof in a visualization system.

26. The method according to claim 25, further comprising moving a bed bearing the patient's body within the treatment room so that the at least one point of intersection with the surface of the patient's body, matches with the target position.

27. The method according to claim 25, further comprising:
calculating at least three points of intersection between at least three lines and the surface of the patient's body, wherein said points of intersection indicate the isocenter of the irradiation device;
displaying the at least three points of intersection in the visualization system;
calculating at least three target positions based on the transformation value; and
displaying the at least three target positions in the visualization system.

28. The method according to claim 25, further comprising:
representing the surface of the patient's body in the visualization system; and
displaying the at least one point of intersection with the surface of the patient's body and the target position on the surface of the patient's body represented in the visualization system.

29. The method according to claim 25, further comprising:
displaying the at least two lines defining the at least one point of intersection indicating the isocenter of the irradiation device in the visualization system; and
displaying the target position of the at least one point of intersection indicating the isocenter of the irradiation device in the visualization system by at least two lines.

30. The method according to claim 25, wherein the reference surface of the patient's body is created as part of radiation planning preceding a positioning of the patient in the treatment room.

31. The method according to claim 25, further comprising:
calculating one or more additional points of intersection with the surface of the patient's body;
determining target positions of the one or more additional points of intersection based of the transformation value; and
displaying the one or more additional points of intersection and the corresponding target positions in the visualization system.

32. The method according to claim 31, wherein the one or more additional points of intersection are points of intersection with edge regions of the surface of the patient.

33. The method according to claim 25, further comprising displaying the at least one point of intersection and the target position thereof from various angles in the visualization system.

34. The method according to claim 25, further comprising verifying the target position of the at least one point of intersection with the surface of the patient's body can be using lasers arranged in the treatment room.

35. The method according to claim 25, further comprising indicating a position of a point of intersection on the surface of the patient's body using lasers arranged in the treatment room, and displaying the position in the visualization system, wherein the position is determined as one of: (i) the target position; or (ii) as an additional target position.

36. A system for supporting a process of positioning a patient in a treatment room for an irradiation process with an irradiation device, the system comprising:
a 3D surface detection system configured to detect a surface of a patient's body;
an evaluation apparatus configured to:
determine at least one point of intersection between at least two lines and the detected surface of the patient's body, wherein said point of intersection indicates an isocenter of the irradiation device;
determining a reference surface of the patient's body indicating a target position of the patient's body relative to the isocenter of the irradiation device;
use the reference surface of the patient's body to calculate a transformation value required to produce a match between the detected surface of the patient's body and the reference surface;
determine a target position of the at least one point of intersection with the detected surface of the patient's body based on the transformation value, wherein the point of intersection indicates the isocenter of the irradiation device; and
a visualization system configured to display the at least one point of intersection with the detected surface of the patient's body and the target position thereof based on the transformation value,
wherein the at least one point of intersection indicates the isocenter of the irradiation device.

37. The system according to claim 36, further comprising a patient bed bearing the patient's body and configured to be moved within the treatment room so that the at least one point of intersection with the detected surface of the patient's body can be matched with the target position.

38. The system according to claim 36, wherein the evaluation apparatus is configured to determine at least three points of intersection between at least three lines and the detected surface of the patient's body and at least three target positions corresponding to each of the at least three points of intersection, wherein the at least three target positions are determined based on the transformation value, and wherein the visualization system is further configured to display the at least three target positions of the at least three points of intersection with the detected surface of the patient's body, wherein the at least three points of intersection indicate the isocenter of the irradiation device.

39. The system according to claim 36, wherein the visualization system is further configured to represent the detected surface of the patient's body, and to display the at least one point of intersection with the detected surface of the patient's body and the target position thereof on the surface of the patient's body represented in the visualization system, wherein the at least one point of intersection indicates the isocenter of the irradiation device.

40. The system according to claim 36, wherein the visualization system is configured to display the at least two lines defining the at least one point of intersection indicating the isocenter of the irradiation device, and is further configured to display the target position of the at least one point of intersection indicating the isocenter of the irradiation device by at least two lines.

41. The system according claim 36, further comprising a radiation planning system configured to create the reference surface of the patient's body as part of radiation planning preceding a positioning of the patient in the treatment room.

42. The system according to claim 36, wherein the evaluation apparatus is further configured to determine at least one additional point of intersection with the detected surface of the patient's body, and wherein the visualization system is further configured to display the at least one additional point of intersection and target position of the at least one additional point of intersection, wherein the target position of the at least one additional point of intersection is determined based on the transformation value.

43. The system according to claim 41, wherein the one or more additional points of intersection are points of intersection with edge regions of the surface of the patient.

44. The system according to claim 36, wherein the visualization system is further configured to display the at least one point of intersection and the target position thereof from various angles.

45. The system according to claim 36, further comprising cross lasers arranged in the treatment room, and wherein the evaluation apparatus is further configured to verify the target position of the at least one point of intersection with the detected surface of the patient's body using the cross lasers, wherein said point of intersection indicates the isocenter of the irradiation device.

46. The system according to claim 42, wherein the evaluation apparatus is further configured to determine at least one additional target position of the at least one additional point of intersection with the surface of the patient's body, and wherein the visualization system is further configured to display the at least one additional point of intersection with the detected surface of the patient's body and the at least one additional target position thereof.

47. The system according to claim 36, further comprising lasers arranged in the treatment room that are configured to indicate a position of a point of intersection on the detected surface of the patient's body, wherein the evaluation apparatus is further configured to determine the indicated position as the target position or as an additional target position, and wherein the visualization system is further configured to display the target position or the additional target position.
